(19) United States
(12) Patent Application Publication
(10) Pub. No.: US 2021/0098110 A1
(43) Pub. Date: Apr. 1, 2021

(54) Digital Health Wellbeing

(71) Applicants: Periyasamy Periyasamy, Fairfax, VA (US); Hariprasad Periyasamy, Fairfax, VA (US); Abhigeetha Periyasamy, Fairfax, VA (US); Jayadeepha Periyasamy, Fairfax, VA (US)

(72) Inventors: Periyasamy Periyasamy, Fairfax, VA (US); Hariprasad Periyasamy, Fairfax, VA (US); Abhigeetha Periyasamy, Fairfax, VA (US); Jayadeepha Periyasamy, Fairfax, VA (US)

(21) Appl. No.: 16/938,086

(22) Filed: Jul. 24, 2020

Related U.S. Application Data
(60) Provisional application No. 62/907,716, filed on Sep. 29, 2019.

(51) Int. Cl. G16H 20/70; G16H 40/67; G16H 10/20; G16H 50/70; G16H 50/20; G06Q 50/00; G16H 50/50; H04L 29/06; A61B 5/00; A61B 5/16; G09B 19/00; A61M 21/02; H04L 12/58

(52) U.S. Cl. CPC G16H 20/70; G06Q 50/265; G16H 10/20; G16H 50/70; G16H 50/20; G06Q 50/01; G16H 50/50; H04L 63/10; A61B 5/4815; A61B 5/165; G09B 19/00; A61B 5/0022; A61B 5/7465; A61M 21/02; A61B 5/7264; A61B 5/7275; A61B 5/167; H04L 51/02; G16H 40/67

(57) ABSTRACT

Apparatus and methods include collecting continuously user data from at least one of the mobile device, a user device, a user, or a public records database; identifying one or more symptoms exhibited by the user based at least in part on the collected user data; determining a mental health condition of the user based at least in part on the identified symptoms, a cognitive behavioral therapy information, and a list of diagnoses based on diagnostic and statistical manual of mental disorders; identifying a root cause of the mental health condition based on the collected user data; and recommending by a virtual agent self-help to the user for managing the mental health condition and the root cause during a one-on-one interactive therapy and counseling session.

### CROSS-REFERENCE TO RELATED APPLICATION

This patent application claims the priority benefit under 35 U.S.C. § 119(e) of U.S. Provisional Application No. 62/907,716, filed on Sep. 29, 2019, the contents of which are herein incorporated by reference in their entirety.

### BACKGROUND OF THE INVENTION

### 1. Field of the Invention

The present invention relates to apparatus, non-transitory computer-readable media, systems, and methods for digital health wellbeing, in particular, to apparatus, non-transitory computer-readable media, systems, and methods for a continuous, real-time monitoring of mental health conditions of a user using personal devices including mobile, smart phones or tables and providing a one-on-one interactive therapy and consulting session with a virtual agent during which the virtual agent can recommend personalized self-help to the user.

### 2. Description of the Related Art

In view of the advanced development and deployment of mobile devices throughout society, e.g., cellular devices, smart phones, etc., and their ability to collect data specific to users, mental health monitoring using those mobile devices have become increasingly popular. Several mobile applications may connect patients to psychiatrists and/or counselors, but may not track and monitor patients continuously, and thus, may fail to alert the patients of potential issues based on their current needs. Many of these digital solutions may focus on offering remedies for mental wellbeing, but may not continuously collect real-time user data, and thus, are unable to offer personalized solutions at the moments the patients are experiencing symptoms of a potential mental health condition or disorder. That is, patients may be showing symptoms of depression, stress, anxiety, or other potential disorder, but there may not be the capability or application to continuously collect real-time health information of the patients, which could potentially enable the system or device to detect the patient's symptoms and identify their potential mental and behavioral illnesses, disorders, or conditions during the onset of the symptoms and potential mental health conditions.

### SUMMARY OF THE INVENTION

A mental health monitoring device in accordance with the present disclosure seeks to provide a solution to the aforementioned problems with the conventional mental health monitoring devices by using a mobile device, e.g., a smart phone, wearable biomarker tracking devices (e.g., a smart watch, smart glasses, etc.), other user devices (e.g., laptop computers, etc.), and public data to continuously collect, track, screen, monitor, or alert patients real-time. As such, data collection may take place via a mobile health application (e.g., implemented in the mobile device) utilizing screen-time, mobile statistics, smart watches, third party applications, or a user input. Third party applications may use mobile statistics from mobile providers (e.g., Apple HealthKit™, Google Fit™, Samsung Health™, etc., and include applications or hardware that also report to the mobile statistics including activity trackers such as Apple Watch™, Fitbit™, Garmin™, etc. Third party applications may also include external sensors like heart rate, glucose monitoring, oxygen saturation level sensor (SPO2), photoplethysmograph (PPG), etc. The tracked and monitored data may be used to predict potential symptoms of mental health conditions (e.g., mental disorders, behavioral illnesses, etc.) and identify the potential mental health conditions at the onset of the symptoms so as to ensure that those mental health conditions are identified for treatment as soon as practicable, and thus do not become exacerbated. The mental health monitoring devices in accordance with the present disclosure may utilize a series of topics predetermined by a patient mental health questionnaire to identify a type and severity of the mental health conditions.

The mental health monitoring devices in accordance with the present disclosure may be directed to users who are going through different facets of potential mental health issues and may provide tailored cognitive behavioral therapies (CBTs) and counseling using a personal mobile device such as a smartphone. A main component of the mental health monitoring application (the “mental health monitoring app”) is an interactive virtual agent, e.g., chatbot. Chatbot is capable of interacting with the user via instant texts, live audio, and/or live video at any time. Every interaction involves a semantic segmentation of the conversations to extract meaningful information from the user. From the segmented conversations, regression models are run in the background to interpret the behavior of the user based on his/her mental health condition(s). The mental health conditions may be categorized and captured using neural networks that may be trained on characteristics and understanding of the different mental health conditions or disorders. Chatbot can also extract vital information (including, but not limited to, sleep data, heartrate, calorie intake (e.g., consumed through food intake and activities), and activities) which may be biomarkers for various mental health conditions or disorders.

Chatbot takes into account the collected data of the user to provide personalized solutions based on the problem at hand in relation to the severity of the illness. Based on the user's previous and current data, e.g., sleep, physical activity, healthy eating, social activity, or mood, an option of visuals (e.g., graphs, charts, statistics, etc.) describing the user data within a specified or predetermined period may promote understanding of the user's lifestyle and a contributing factor to the user's mental health condition. In addition to the tracked data, the mental health monitoring device may utilize the patient health questionnaire to determine the user's current or potential mental health conditions, e.g., stress, anxiety, depression, mental disorder, behavioral disorder, etc. The mental health monitoring devices in accordance with the present disclosure may continuously track, monitor, screen, and alert the user based on the severity of the current or potential mental health conditions, while chatbot may provide personalized solutions correlating to the determined potential mental health condition or predicament the user may be facing. A root cause may include a contributing factor, and the root cause to the potential mental health condition may be identified by continuously monitoring the data collected from the tracked user data and user inputs. Chatbot may provide one-on-one interactive therapy and counseling sessions to provide self-help to manage the identified symptom and/or potential mental health conditions, e.g., digital therapeutics.

Further, the mental health monitoring devices in accordance with the present disclosure also provides a low interference privacy protection schemes when communicating via, e.g., cloud servers with untrusted memory. For example, all the data interactions may be encrypted and take place in trusted private clouds.

Accordingly, it is an object of the present disclosure to provide novel systems, apparatus and methods that not only collect data from various data sources, e.g., mobile health application, screen time, third party applications, public data, and user input in order to continuously track, monitor, screen, and identify the user's potential mental health condition(s), e.g., stress, anxiety, and depression, but also provide personalized, interactive, and timely recommendations or solutions to the identified potential mental health conditions of the user by means of digital therapy and counseling with a virtual agent, e.g., a chatbot, at the on-set of recognized and identified symptoms or contributing factors.

Such an object may be achieved in accordance with the present disclosure by providing methods for monitoring mental health using a mobile device. The methods may include: collecting continuously user data from at least one of the mobile device, a user device, a user, or a public records database, where the user data may include: user input, public records including family relations data, and/or a plurality of data tracked by the mobile device or the user device which can include sleep data, activity data, mood data, screen time data, social data, or food data; identifying one or more symptoms exhibited by the user based at least in part on the collected user data; determining a mental health condition of the user based at least in part on the identified symptoms, selecting cognitive behavioral therapy information, and a list of diagnoses based on diagnostic and statistical manual of mental disorders; identifying a root cause or a contributing factor for the mental health condition based at least in part on the collected user data; and recommending via a virtual agent self-help to the user for managing the mental health condition, the root cause, and/or the contributing factor(s) during a one-on-one interactive therapy and counseling session with the virtual agent.

In some examples, continuously collecting user data may include implementing a mental health monitoring application in the mobile device; receiving an authorization from the user to access the user data from the mobile device and the user device; and providing a mental health questionnaire including a plurality of questions customized to the collected user data and tailored to obtain information related to the mental health condition of the user, the information including a type or severity of the mental health condition.

In some examples, the virtual agent may recommend self-help to the user which may include directing the user to a mental health expert for a granular diagnosis and/or therapy in an online platform via the mental health monitoring application.

In some examples, the mental health questionnaire may use a patient health questionnaire—9 module.

In some examples, the user devices may include a wearable device for monitoring or tracking at least one of the tracked data.

In some examples, the virtual agent may be a chatbot utilizing at least one of an artificial intelligence (AI) based dialogue conversational engine, AI based mental health and wellbeing question engine, or AI based mental health and wellbeing response engine.

In some examples, the virtual agent may recommend self-help to the user which may include at least one of a text therapy and counseling session, a live audio therapy and counseling session, or a live video therapy and counseling session with the chatbot.

In some examples, the virtual agent may recommend self-help to the user by displaying on a display screen of the mobile device a page for each of the tracked data.

In some examples, the virtual agent may recommend self-help to the user by displaying on a display screen of the mobile device a page for recommended self-help including meditation, supportive therapy for the contributing factor or the root cause related to the diagnosed mental health condition, relaxation techniques, or an audio session for inducing positive thoughts, peace, or stress relief.

In some examples, determining the mental health condition of the user may include determining a level of severity of the mental health condition, where the level of severity comprises a low risk, a medium risk, or a high risk; and alerting at least one of the user or a mental health expert of the mental health condition based at least in part on a determination that the level of severity is at the high risk, immediately upon the determination.

In some examples, a mood of the user may be determined based on the mood data using one or more AI facial recognition model, AI voice based model, or AI text based sentiment model.

In some examples, the social data may be used to determine the mental health condition of the user based at least in part on a time spent by the user on a social media application, a phone, or the mental health monitoring device.

In some examples, an AI based prediction of a future mental health condition of the user may be made based on food habits of the user.

Apparatus for monitoring mental health are provided. The apparatus may include a processor, memory in electronic communication with the processor, and instructions stored in the memory and executable by the processor to cause the processor to: continuously collect user data from at least one of the mobile device, a user device, a user, or a public records database, where the user data may include a user input, public records including family relations data, or a plurality of data tracked by the mobile device or the user device including sleep data, activity data, mood data, screen time data, social data, or food data; identify one or more symptoms exhibited by the user based at least in part on the collected user data; determine a mental health condition of the user based at least in part on the identified symptoms, a cognitive behavioral therapy information, and a list of diagnoses based on diagnostic and statistical manual of mental disorders; identify a root cause of the mental health condition based at least in part on the collected user data; and recommend, by a virtual agent, self-help to the user for managing the mental health condition, the root cause, or the contributing factor during a one-on-one interactive therapy and counseling session.

In some examples, the instructions cause the processor to collect continuously user data may include instructions for the processor to implement a mental health monitoring application in the mobile device; receive an authorization from the user to access the user data from the mobile device and the user device; and provide a mental health questionnaire including a plurality of questions customized to the collected user data and tailored to obtain information related to the mental health condition of the user, the information including a type or severity of the mental health condition.

In some examples, the instruction to cause the processor recommend, by the virtual agent, self-help to the user session may include an instruction to direct the user to a mental health expert for a granular diagnosis and therapy in an online platform via the mental health monitoring application.

In some examples, the virtual agent is a chatbot utilizing at least one of an AI based dialogue conversational engine, AI based mental health and wellbeing question engine, or AI based mental health and wellbeing response engine.

In some examples, the one-on-one therapy and counseling session may include at least one of a text only therapy and counseling session, a live audio therapy and counseling session, or a live video therapy and counseling session with the chatbot.

In some examples, the instructions to cause the processor to determine the mental health condition of the user comprises instructions to cause the processor to: determine a level of severity of the mental health condition, wherein the level of severity comprises a low risk, a medium risk, or a high risk; and alert at least one of the user or a mental health expert of the mental health condition based at least in part on a determination that the level of severity is at the high risk, immediately upon the determining.

Non-transitory computer-readable media storing code for monitoring mental health are provided. The code may include instructions executable by a processor to: collect continuously user data from at least one of the mobile device, a user device, a user, or a public records database, where the user data may include a user input, public records including family relations data, or a plurality of data tracked by the mobile device or the user device including sleep data, activity data, mood data, screen time data, social data, or food data; identify one or more symptoms exhibited by the user based at least in part on the collected user data; determine a mental health condition of the user based at least in part on the identified symptoms, a cognitive behavioral therapy information, and a list of diagnoses based on diagnostic and statistical manual of mental disorders; identify a root cause of the mental health condition based at least in part on the collected user data; and recommend, by a virtual agent, self-help to the user for managing the mental health condition, the root cause, or the contributing factor during a one-on-one interactive therapy and counseling session.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1 is a schematic diagram showing an example mental health monitoring system in accordance with the present disclosure;

FIG. 1A is a block diagram of an example mental health monitoring application in accordance with the present disclosure;

FIG. 2 is a block diagram of an example mental health monitoring device in accordance with the present disclosure;

FIGS. 3A-B are example screen shots of a mental health monitoring device in accordance with the present disclosure;

FIGS. 4A-B are another example screen shots of a mental health monitoring device in accordance with the present disclosure;

FIG. 5 is yet another example screen shot of a mental health monitoring device in accordance with the present disclosure; and

FIG. 6 is yet another example screen shot of a mental health monitoring device in accordance with the present disclosure;

FIG. 7 is yet another example screen shot of a mental health monitoring device in accordance with the present disclosure; and

FIG. 8 is a flow chart for a method for monitoring mental health in accordance with the present disclosure.

### DETAILED DESCRIPTION OF EXEMPLARY EMBODIMENTS

As used herein, the statement that two or more parts or components are “coupled” shall mean that the parts are joined or operate together either directly or indirectly, i.e., through one or more intermediate parts or components, so long as a link occurs.

As used herein, “directly coupled” means that two elements are directly in contact with each other.

As used herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

As described in greater detail herein in connection with various particular embodiments, the disclosed concept provides a novel mental health monitoring system, apparatus, or system using a mobile device.

The disclosed concept will now be described, for purposes of explanation, in connection with numerous specific details in order to provide a thorough understanding of the subject innovation. It will be evident, however, that the disclosed concept can be practiced without these specific details without departing from the spirit and scope of this innovation.

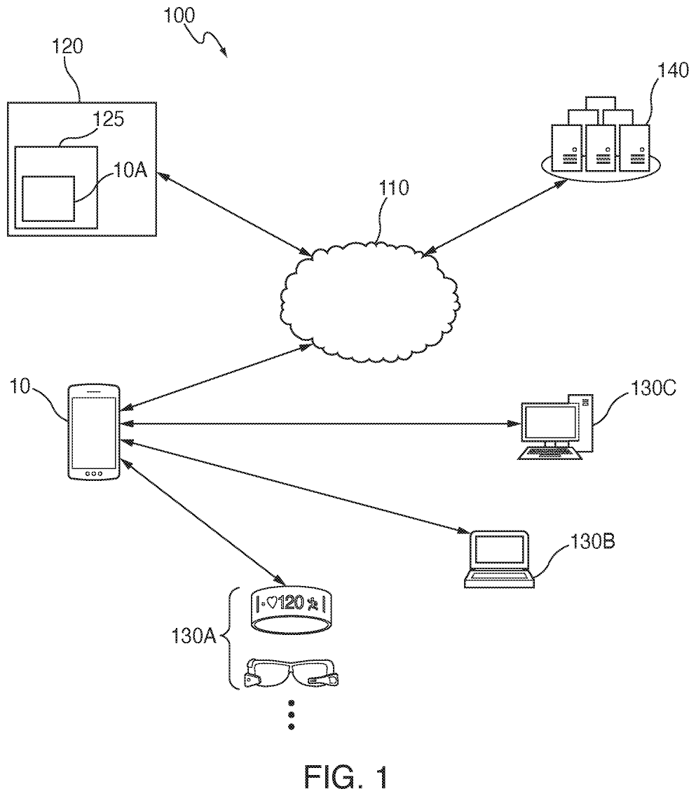

FIG. 1 is a diagram showing an example mental health monitoring system 100 for continuous, real-time monitoring, tracking, and determining the user's mental health conditions via, e.g., a cloud network 110 in accordance with the present disclosure. The system 100 includes a cloud network 110 via which a server 120, a mental health monitoring device 10, user devices 130A, B, and C, and public records database 140 may communicate with one another remotely and/or wirelessly. The server 120 may be a main or regional server for the mental health monitoring system 100 and include, among others, memory 125 for storing the user data including a user input, e.g., membership information, emergency contacts, contact information for a mental health expert (e.g., a psychotherapist, psychologist, etc.), mental health information collected by the mental health monitoring device 10 from the user devices 130A,B,C, and the public records collected from a public records database 140. The memory 125 may include various software, firmware and/or hardware programs (e.g., the mental health monitoring app 10A to be downloaded to a mobile device such as a smart phone), applications, and updates for the operations of the mental health monitoring system 100. The server 120 may also provide any other functions necessary to continuously and effectively operate the system 100. For example, the server 120 may perform data analysis and diagnostics based on the collected data by the mental health monitoring device 10 according to, e.g., a neural network, where conducting such data analysis and diagnostics may use too much battery power for the mental health monitoring device 10. The server 120 may then transmit result of the data analysis and diagnosis to the mental health monitoring device 10. In another example, the server 120 may provide alert capabilities in case of a possible emergency, e.g., a possible heart attack, a suicide attempt, a drug overdose, etc., where the mental health monitoring device 10 may not be capable of making such alert (e.g., being turned off or out of battery).

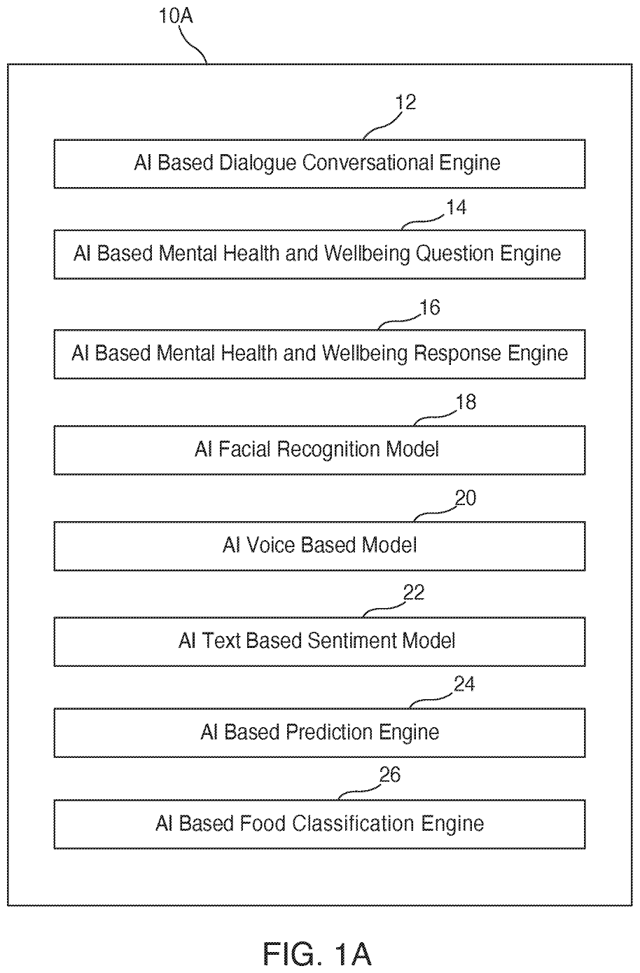

The mental health monitoring device 10 may be a user equipment that is mobile, portable, and capable of performing operations, including but not limited to, communicating with other user devices 130A,B,C, collecting continuously user data from at least one of the mobile device, a user device, a user, or a public records database, where the user data includes a user input, public records including family relations data, or a plurality of data tracked by the mobile device or the user device including sleep data, activity data, mood data, screen time data, social data, or food data; identifying one or more symptoms exhibited by the user based at least in part on the collected user data; determining or diagnosing a potential mental health condition of the user based at least in part on the identified symptom, a cognitive behavioral therapy information, and a list of diagnoses based on diagnostic and statistical manual of mental disorders; identifying a root cause or a contributing factor for the mental health condition based at least in part on the collected user data; and recommending by a virtual agent self-help to the user for managing the mental health condition, the root cause, or the contributing factor during a one-on-one interactive therapy and counseling session with the virtual agent. While FIG. 1 shows the mental health monitoring system 10 being a smartphone, it will be understood by the person of ordinary skill in the art that the mental health monitoring device 10 may be any mobile user devices, for example, a cellular phone, a personal digital assistant (PDA), a wireless communication device, a handheld device, or the like, that are capable of performing the previously stated operations of the mental health monitoring device 10. Examples of the user devices 130A, B, C may include wearable devices 130A such as smartwatches, smart glasses, trackers, sensors, etc., and nonwearable devices such as a tablet computer, a laptop computer 130B, a cordless phone, a personal electronic device, a handheld device, a personal computer 130C, an Internet of Things (IoT) device, an Internet of Everything (IoE) device, a machine type communication (MTC) device, an appliance, an automobile, or the like, that may track, monitor, and collect user data. For example, the wearable devices 130A may be a smart watch that monitors and tracks, for example, the user's biomarkers such as heart rate and physical activities (e.g., a number of steps taken, miles run, stairs climbed, etc.), or smart glasses that monitor the user's brain rhythms, mood, anxiety levels, etc. In some examples, the nonwearable user devices may be a laptop computer 130B or a personal computer 130C, which the user uses to perform work, engage in social media, watch a movie, listen to music, play a game, etc. while the user is not using the mental health monitoring device (e.g., the smartphone) 10. The user data detailing the user's activities using the laptop computer 130B or the personal computer 130C may be communicated to the mental health monitoring device 10 via automatic synchronization or uploaded by the user using a cloud storage or a flash drive. In some other examples, the non-wearable user devices may include an automobile that includes a tracker or sensor of the user's heartrate, brain rhythm, pupil conditions, etc. The trackers or sensors may transmit the user data that they monitor to the mental health monitoring device 10. The user devices 130A,B, and C may communicate the user information to the mental health monitoring device 10 via wired or wireless communication link, using Bluetooth,™ WiFi Direct, Zigbee™, or near field communication such as radio-frequency identification technologies. The public record database 140 may include, e.g., family relation information of the user, driver's license records, neighborhood information of the user's current location, etc. Since such data may be publically available, the mental health monitoring device 10 may be able to access and store such data as necessary for its operations. The mental health monitoring device 10, its operations, and its benefits will be explained in further detail with reference to FIG. 2.

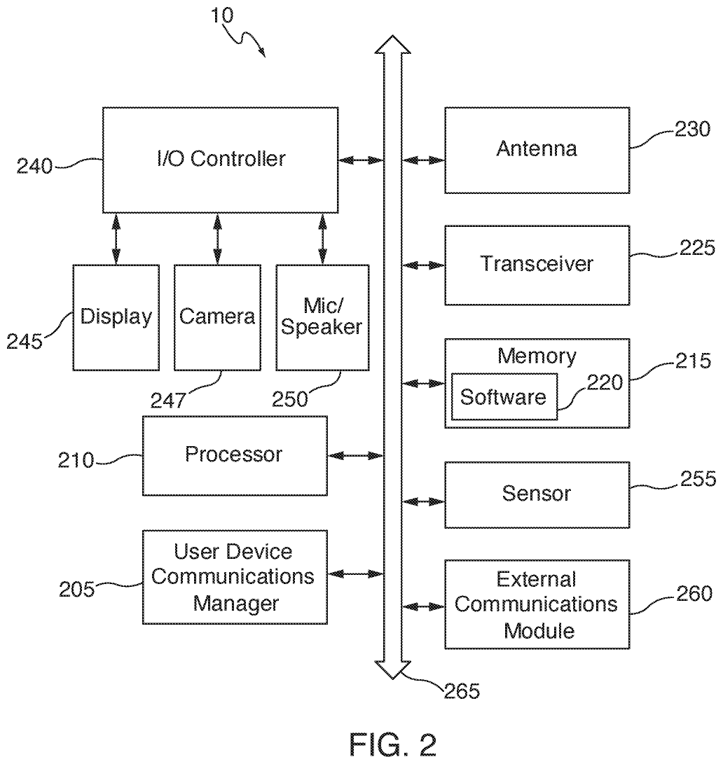

FIG. 1A is a block diagram of an example mental health monitoring app 10A in accordance with the present disclosure. In order to provide an accurate, reliable, effective and humanized interactions and identification of a potential mental health condition of the user, the mental health monitoring app 10A may utilize a neural network, e.g., artificial intelligence (AI) based dialogue conversational engine 12, AI based mental health and wellbeing question engine 14, AI based mental health and wellbeing response engine 16, AI facial recognition model 18, AI voice based model 20, AI text based sentiment model 22, an AI based prediction of a future health condition 24, an AI based food classification engine 26, etc. For example, the one-on-one interactive personalized therapy and counseling session (e.g., a live therapy and counseling session via text 300A, a live audio therapy and counseling session, a live video therapy and counseling session 300B with a chatbot as shown in FIGS. 3A and 3B) may utilize AI based dialogue conversational engine 12, AI based mental health and wellbeing question engine 14, or AI based mental health and wellbeing response engine 16, so that the virtual agent, e.g., the chatbot, may conduct the therapy and counseling session as if the user is having the session with a trained human mental health personnel (e.g., psychotherapist, psychologist, etc.). The AI based dialogue conversational engine 12 allows the Chabot to have a conversational dialogue with the user based on user's dialogue style, preferences, or habits observed by the mental health condition device 10. The AI based mental health and wellbeing question engine 14 allows the a chatbot to ask appropriate questions to the user based on the collected data, identified symptoms, and determined mental health condition of the user. The AI based mental health and wellbeing response engine 16 allows the chatbot to respond to the user when interacting with the user, especially during the one-on-one interactive therapy and counseling session. In another example, the AI facial recognition model 18, AI voice based model 20, or AI text based sentiment model 22 may be utilized to determine the user's mood. In yet another example, the AI based prediction engine 24 may be used to determine, e.g., the user's future health condition based at least in part on the user's past or current food habits (e.g., eating habits, drinking habits, etc.). In yet another example, the AI based food classification engine 26 may be used to determine what type of food the user is intaking (e.g., based on a picture the user uploads to the mental health monitoring device 10 via a camera). These AI based engines and models may utilize a cognitive behavioral therapy information and/or training modules, a list of diagnoses based on diagnostic and statistical manual of mental disorders, mental health condition treatment and preventive measure training modules, so as to enable the chatbot to engage in a meaningful, effective, reliable, accurate, timely, and humanized interactions with the user at all time, especially, the one-on-one interactive therapy and counseling session with the user. It will be understood by the person of ordinary skill in the art that FIG. 1A is not limiting and for illustrative purposes only, thus may include or use different AI based engines or models that may enhance or amplify the efficacy, accuracy, and reliability of the operations and functions of the mental health monitoring device 10.

FIG. 2 is a block diagram of a mental health monitoring device 10 according to one particular, non-limiting exemplary embodiment, including the various components housed therein. The mental health monitoring device 10 may be an example of a user equipment such as a smartphone as described above. The mental health monitoring device 10 may include, but not limited to, user device communications manager 205, processor 210, memory 215, software 220, transceiver 225, antenna 230, I/O controller 240, display 245, audio devices 250 (e.g., a microphone, speaker, etc.), a sensor 255 (e.g., accelerometer, gyroscope, pressure sensor, thermal sensor, Hall-effect sensor, etc.), and an external communications module 260 (e.g., Application Programming Interface, etc.). These components may be in electronic communication via one or more buses (e.g., bus 265). The mental health monitoring device 10 may communicate wirelessly with the server 120 and the public records database 140, or communicate via wired or wireless links with the user devices 130A, B, and C.

In general, the user may utilize the mental health monitoring device 10 by first downloading a mental health monitoring app 10A (e.g., MyLifeCoach (MLC) app) from, e.g., Google Play™ or Apple's App Store™ and installing the mental health monitoring app on his/her mobile device (e.g., a smartphone). The mental health monitoring app 10A may utilize a neural network, e.g., artificial intelligence (AI) based dialogue conversational engine 12, AI based mental health and wellbeing question engine 14, AI based mental health and wellbeing response engine 16, AI facial recognition model 18, AI voice based model 20, AI text based sentiment model 22, an AI based prediction engine 24, etc.

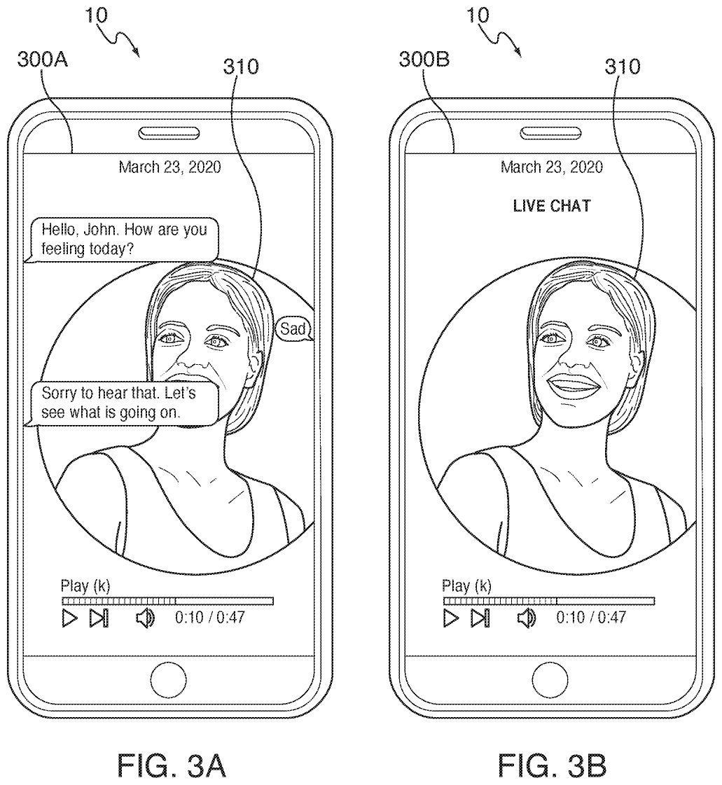

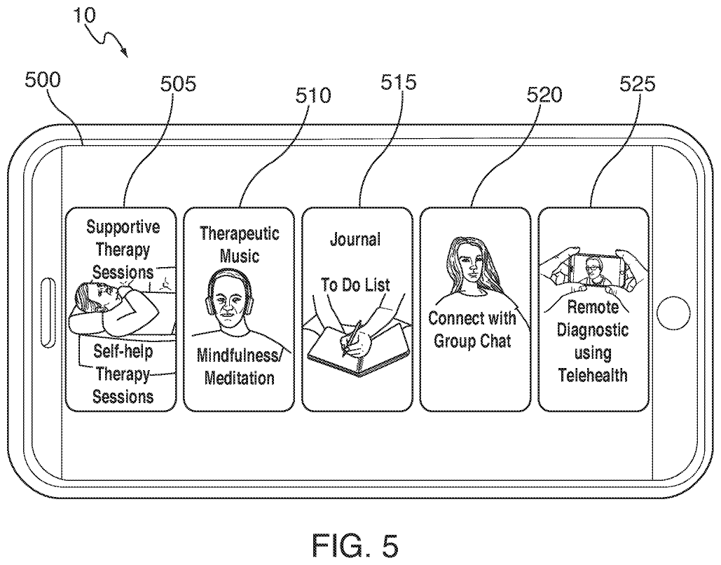

Upon installing the mental health monitoring app 10A to the mental health monitoring device 10, the user may sign in as a guest or via his/her MLC, Facebook™ or Google™ accounts. When the user signs in as a guest, the user enjoys the app's features with some limitations. The user may access a main dashboard (e.g., the main dashboard 400 as shown in FIG. 4A) and subpages for various monitored items (e.g., sleep 405, physical activity 410, mood 415, food 420, work 425, and social 430 as shown in FIG. 4B) with sample data. For example, the food intake of the user may be monitored and tracked by, e.g., the user uploading pictures and viewing his/her food intake under a food subpage. Further, based on the monitored items, the user may view a sample recommendation plan 600 as shown in FIG. 6, add to a “To Do” list or utilize a journal entry section 420. However, the user may only access some cognitive behavioral therapy (CBT) files (e.g., self-help, supportive therapy, relaxation techniques and music) while all other files may be locked in a wellbeing screen. Also, the user may not be able to designate CBT files as favorite. Additionally, the user may not send feedback through a support menu or access group chat facilities. While the user may chat with the chatbot by, e.g., text with limited access, the user may not access a live one-on-one interactive video and/or audio therapy and counseling session with the chatbot.

When the user logs in with an MLC, Google™, Facebook™ account, or any other account that offers private access to the mental health monitoring app 10A, the user may enjoy all available features of the mental health monitoring app 10A via the mental health monitoring device 10 without the restrictions mentioned above. In order to enjoy the features of the mental health monitoring app 10A, the user may first approve his/her health data access and any wearable data access to, e.g., Fitbit™ or Apple Watch™. When the user logs in to the mental health monitoring app 10A for the first time, the mental health monitoring app 10A may collect telemetric data for a predetermined period (e.g., a 90-day period) from, e.g., HealthKit™, Fitbit™, Apple Watch™, etc. The telemetric data may include social data 430, including mobile network usage (e.g., a phone usage, a social media usage, an Internet usage) and screen time, where each app's usage time may be recorded. The telemetric data may be displayed on a subpage for social data 420 so that the user may be able to see how much time he/she spends on the social media, the phone, the mental health monitoring app 10A, etc. At the user's subsequent log-ins, the telemetric data collection time may vary, e.g., one-day, one-week, one-month, etc.

When the user logs in, the landing page shows a chatbot (e.g., a chatbot 310 as shown in FIG. 3B). The chatbot 310 may be called Aura or any other suitable name determined by the app provider or the user. The chatbot 310 may greet the user with the user's username. During the first login, the chatbot 310 may play an informative video that explains the mental health monitoring app's features by going through a number of webpages, e.g., a main dashboard 400, subpages for each monitored and tracked conditions (e.g., a sleep data subpage 405A as shown in FIG. 4B), subpages for recommended self-help, etc. The main dashboard 400 may show the user data monitored and tracked by the mental health monitoring device 10. The user data displayed on the main dashboard 400 may include the user's daily, weekly, and monthly sleep data 405, activity data 410, mood data 415, food data 420, work data 425, or social data 430 in one screen. It will be understood by the person of ordinary skill in the art that FIG. 4A is not limiting and for illustrative purposes only, and may include different items being monitored or tracked by the mental health monitoring device 10.

Each of the monitored and tracked user data may include a link that takes the user to a subpage detailing the monitored and tracked user data upon user's command via, e.g., a touch, click, voice, etc. For example, a subpage 405A for the user's sleep data as shown in FIG. 4B may illustrate details 405B of the user's sleep record, e.g., time during which the user was asleep, heart rates, breathing rates, a number of bathroom visits, and a graph 405C showing the quality of sleep while the user was asleep. In some cases, the sleep subpage 405A may also include a link to another subpage that tracks the details of the user's sleep for different periods, e.g., a week, a month, a three-month, etc. For example, the graph 405C showing the quality of sleep may have a link or itself be a link to another subpage that shows the statistical and graphical data illustrating the quality of sleep over a various predetermined period (e.g., a day, a week, a month, a 90-day period, etc.) in detail so that the user may be able to see his sleep data over different periods and ascertain what may have triggered his current mental health condition based on the statistical and graphical sleep data. In such examples, upon a user command, e.g., a touch, a click, or a voice command, the link to the statistical and graphical data over different periods may be triggered to display the statistical and graphical data subpage. That is, the user may touch a sleep data 405 displayed on the main dashboard 400, which then takes the user to a subpage 405A detailing the sleep detail 405B,C of the user during a pre-determined period. Upon another user command (e.g., a touch, a click, a voice command, etc.), a subpage detailing the quality of sleep over different periods via graphs, charts, and/or statistics may be displayed on the screen of the mental health monitoring device 10.

The user data may be collected by the mental health monitoring device 10 itself, wearable devices 130A that monitor and track biomarkers and/or potential mental health condition of the user throughout a pre-determined period, e.g., a day, a week, a month, 90-days, etc., and nonwearable devices 130B,C that may monitor screen time for the user over the predetermined period (e.g., 20 hours on social media over the week of Jun. 28, 2020).

After displaying the subpages, the informative video may show the benefits and a variety of self-help available to the user via the mental health monitoring device 10. The benefits of the mental health monitoring device 10 may include continuous collection of the real-time user data while the user is performing his/her daily activities throughout the day without interrupting the user's daily activities. Thus, no important user data is missed or uncollected by the mental health monitoring device 10, thereby allowing the mental health monitoring device 10 to provide an accurate, reliable, and up-to-date identification of a symptom(s) and determination of a type and severity of a potential mental health condition. Further, with the 24/7 ready and available virtual agent, e.g., a chatbot 310, the user may enjoy timely, and comprehensive recommendation for a variety of self-help designed to deal with the specific symptoms and potential mental health condition identified, where the variety of self-help is available at the user's fingertip or voice. In addition, the chatbot 310 utilizes at least one of AI based dialogue conversational engine 12, AI based mental health and wellbeing question engine 14, or AI based mental health and wellbeing response engine 16, thereby capable of engaging with the user in a personalized, interactive one-on-one therapy and counseling session appropriate for the user's current or potential mental health condition. Moreover, the therapy and counseling session is available via text 300A, live audio and/or live video 300B directly interacting with the chatbot 310, thereby removing possible feelings of isolation or loneliness and providing the immediate and timely advice and counsel when the user needs it most. In some examples, the chatbot 310 may direct the user to a mental health expert for a granular diagnosis and therapy in an online platform via the mental health monitoring application, so that the user may immediately receive an even more detailed determination of the root causes and the mental health condition, cognitive behavioral therapy, self-help recommendations that are personalized and unique to the user at that very instance. In some examples, the chatbot 310 may alert the user or initiate the therapy and counseling session with the user if the severity of the current or potential mental health condition is determined to have a high risk to the user or others around the user—the severity may be categorized as a low risk, a medium risk, or a high risk. If the user is not reachable or responsive, the chatbot may alert a mental health expert (e.g., the user's regular therapist or psychologist, or any mental health expert near the user's current location) or even contact the police for help.

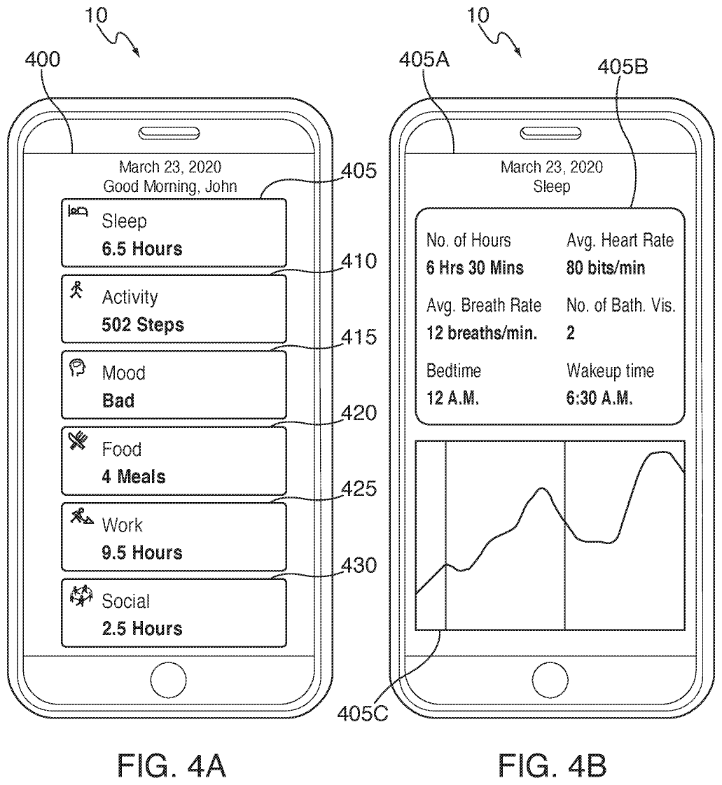

The variety of self-help may be displayed in a comprehensive wellbeing subpage (e.g., the comprehensive wellbeing subpage 500 as shown in FIG. 5). The comprehensive wellbeing subpage 500 may include a variety of self-help, for example, supportive therapy session 505 (e.g., a self-help therapy session), therapeutic music 510 (e.g., mindfulness, meditation, or self-help video or audio), journal 515 with a To-Do list, a group chat connection 520, and a telehealth feature 525. Every positive activity may have incentive points for, e.g., providing motivation to the user and help boost his/her dopamine levels to stay fit, active, and/or healthy by achieving higher incentive numbers and characters. The aggregated reward points may be available in the main dashboard 400.

The supportive therapy session 505 may be provided to cope with or manage the root cause and/or the contributing factor related to the determined mental health condition, and include the personalized, interactive, one-on-one therapy and counseling session with the chatbot 310 via text 300A, audio and/or video 300B over the mental health monitoring device 10. In some examples, the supportive therapy session 505 may display a recommendation plan or list 600 of self-help targeted to overcome the identified symptoms and potential mental health condition, such that the user may follow the list and self-treat him/herself at the on-set of the symptoms and prevent further deterioration of his/her mental health. The list 600 may suggest the user to take a walk for a period, perform breathing exercises, in-take healthy or remedial food, etc.

The therapeutic music 510 may offer relaxation techniques (e.g., breathing, yoga, etc.) or induce positive thoughts, peace, or stress relief. The therapeutic music 515 may be displayed in a subpage including a list of therapeutic music that may be effective in dealing with the symptom(s) and potential mental health condition. The list may have a link to each of the recommended therapeutic music and for example, direct the user to the therapeutic music of the user's choice. The therapeutic music 510 may include music helping the user with his/her sleep (e.g., music for falling asleep, deep sleep, relaxation, inducing sleep, etc.). The therapeutic music 510 may include music helping the user with the user's focus, analytical thinking, problem solving, concentration, etc. Such therapeutic music 410 may provide the user with accelerated learning, healing, stress/pain relief, peace, calming anxiety, positive thoughts, energy boost, motivation to perform certain activities, exercise, meditation, help with memory recall or activating brain, etc. The therapeutic music 410 may include an audio/video self-help session during which the user may view high quality positive thought inducing, peaceful, and motivational photos with the parallax effect. The user may tag the user's favorite audio file, which may appear in the user's library.

The journal 515 may be offered to allow the user a moment to reflect on the day's events and activities and record the user's unfiltered, raw feelings and thoughts on the events and activities, thereby enabling the user to release bottled-up emotions and stresses where it is secure (e.g., no one may access the journal without the user's authorization). The journal 515 may include a To-Do list for the user, which the user may add, edit, or revise as the user see fits. The To-Do list may be similar to the self-help recommendation plan 600 include activities that may benefit the user in overcoming the identified symptom and potential mental health condition, e.g., get up at 6 a.m., call your mom for a chat, meditate, etc.

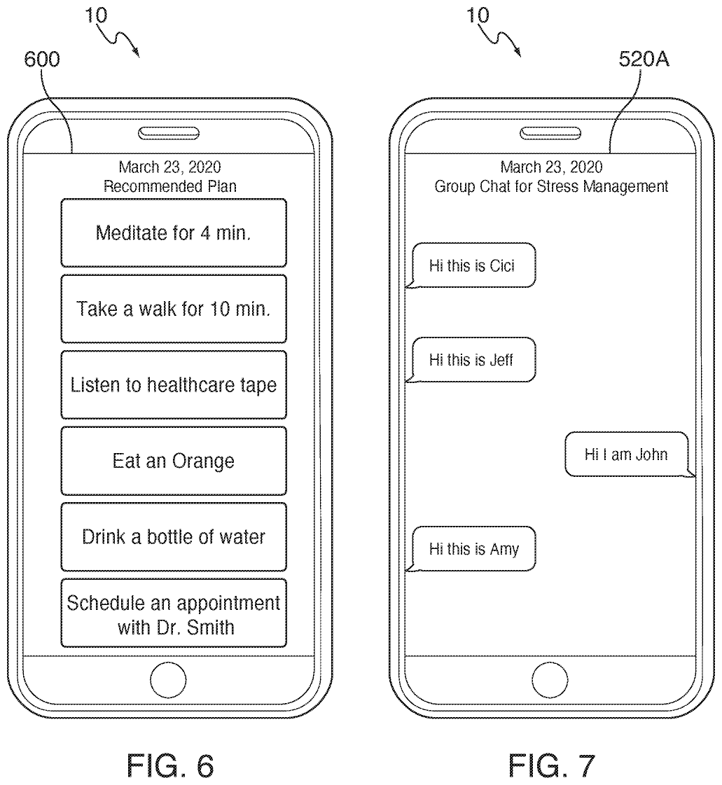

The group chat connection 520 may include a variety of online group chats available dealing with a specific symptom or mental health condition, and a separate channel for each of these online group chats with respect to a specific mental health condition or disorder. For example, there may a channel for a stress group chat (e.g., the stress management group chat 520A as shown in FIG. 7), an anxiety group chat, an eating disorder group chat, a depression group chat, etc. Each of these channels is vetted by AI based engines of the mental health monitoring device 10 in order to ensure that channels available are remedial, and not possibly destructive (e.g., a suicide group chat which encourages suicide, etc.).

The telehealth feature 525 may provide a remote diagnosis from, e.g., a mental health expert over the online platform, via a telehealth app which may be directed by a link on 525. The mental health expert may be the user's own mental health physician, or a mental health expert available when a request for a telehealth session is made by the user.

Therefore, the mental health monitoring device 10 in accordance with the present disclosure not only provides accurate and reliable analysis and determination of a symptom(s) and potential mental health condition based on the comprehensive and real-time user data continuously collected from all available user device tracking the user's biomarkers and/or mental health condition, but also provides a life-saving personalized, interactive, live therapy and counseling when the user needs it the most so as to prevent any unfortunate incidents and allow the user to deal directly with the root cause and/or the contributing factor so as to avoid situations that trigger any mental episodes or breakdowns in the future.

Referring back to FIG. 2, the user device communications manager 205, or its components may be implemented in hardware, software executed by the processor 210, firmware, or any combination thereof. If implemented in software executed by the processor 210, the functions of the user device communications manager 205 may be executed by a general-purpose processor, a digital signal processor (DSP), an application-specific integrated circuit (ASIC), a field-programmable gate array (FPGA) or other programmable logic device, discrete gate or transistor logic, discrete hardware components, or any combination thereof designed to perform various functions, e.g., selecting appropriate user devices 130A, B, and C to communicate with, conveying information indicating parameters of communication layers, determining communication format (e.g., LTE, LTE-A, New Radio, etc.), generating messages in the determined format to communicate with the selected user devices, the server 120, and the public records database 140, transmitting and receiving messages to and from the user devices 130A, B, and C, the server 120, and the public records database 140.

The Processor 210 may include an intelligent hardware device (e.g., a general-purpose processor, a DSP, a central processing unit (CPUI), a microcontroller, an ASIC, an FPGA, a programmable logic device, a discrete gate or transistor logic component, a discrete hardware component, or any combination thereof). Processor 215 may be configured to execute computer-readable instructions 220 stored in a memory 215 to perform various functions. The computer-readable instructions 220 may be implemented from an external software application server via external communications module 260.

Memory 215 may include random access memory (RAM) and read only memory (ROM). The memory 215 may store computer-readable, computer-executable software 220 including instructions, that when executed, cause the processor 210 to perform various functions described herein. In some cases, the memory 215 may contain, among other things, a basic input/output system (BIOS) which may control basic hardware or software operation such as the interaction with peripheral components or devices.

Software 220 may include code or algorithm to implement aspects of the present disclosure. For example, software 220 may include instructions for the processor 210 to perform at least: implement (e.g., install) a mental health monitoring software application upon a prompt by the user via external communication module 260; display on a screen of the mental health monitoring device 10 a plurality of webpages including a main dashboard 400 for providing general information of the mental health monitoring device 10, sub-pages displaying various user data being monitored and tracked by the mental health monitoring device 10 itself and the user devices 130A, B, and C, sub-pages showing a list of self-help recommended and each of the recommended self-help; provide sub-pages allowing the user to access various user data being monitored; continuously collect user data from at least one of the user devices 130A, B, and C, public records database 140, etc.; analyze the user data based at least in part on user input (e.g., answers to a health questionnaire, real-time user responses to questions posed by the virtual agent) and expert mental health guidelines (e.g., expert mental health diagnostic guidelines, expert mental health therapy and counseling guidelines, etc.); identify one or more symptoms exhibited by the user based at least in part on the collected user data; determine a potential mental health condition of the user based at least in part on the identified symptoms, a cognitive behavioral therapy information, and a list of diagnoses based on diagnostic and statistical manual of mental disorders; identify a root cause or a contributing factor for the potential mental health condition based at least in part on the collected user data; provide a one-on-one interactive therapy and counseling session with a virtual agent, e.g., a chatbot; recommend by the virtual agent self-help to the user for managing the potential mental health conditions, the root cause, or the contributing factor during the therapy and counseling session; and provide a mental health questionnaire including a plurality of questions customized to the collected user data and tailored to obtain information related to the mental health condition of the user, the information including a type or severity of the mental health condition and store the information in the memory 215. The user data includes, but not limited to, a user input, public records including family relations data, or a plurality of data tracked by the mental health monitoring device 10 or the user device 130A,B,C including, but not limited to, sleep data 405, activity data 410, mood data 415, food data 420, work data 425, and social data 430.

Software 220 may be stored in a non-transitory computer-readable medium such as system memory or other memory. In some cases, the software 220 may not be directly executable by the processor 210 but may cause a computer (e.g., when compiled and executed) to perform functions described herein. The memory 215 may also include firmware embedded for implementing the operations of the mental health monitoring device 10. Transceiver 225 may communicate bi-directionally, via one or more antennas 230, wired, or wireless links. For example, the transceiver 225 may represent a wireless transceiver and may communicate bi-directionally with another wireless transceiver. The mental health monitoring device 10 device may include one or more antennas 230, which may be capable of concurrently transmitting or receiving multiple wireless transmissions, e.g., from the user devices 130A, B, and C. I/O controller 240 may manage input and output signals for the mental health monitoring device 10. In some case, I/O controller 240 may utilize an operation system such as iOS®, ADROID®, MS-DOS®, MS-WINDOWS®, OS/2®, UNITX®, LINUX®, or other known operating system. In other cases I/O controller may be implemented as part of a processor. In some cases, a user may interact with the mental health monitoring device 10 via I/O controller 240 or via hardware components controlled by I/O controller 240, for example, display 245, camera 247, and audio device 250 (e.g., speaker, microphone, etc.). Sensors 255 may include accelerometer, gyroscope, pressure sensor, thermal sensor, Hall-effect sensor, etc., and detect the user's location, activities, or biomarkers such as heartrate. External communication modules 260 may include an application programming interface.

FIG. 3A-B illustrate example screen shots of an interactive one-on-one therapy and counseling session 300A,B of the mental health monitoring device 10 in accordance with the present disclosure. FIG. 3A is a screen shot 300A showing an interactive one-on-one therapy and counseling session with the chatbot via text in accordance with the present disclosure. FIG. 3B is a screen shot 300B showing a live interactive one-on-one therapy and counseling session with the chatbot via video in accordance with the present disclosure. The therapy and counseling session may be via various other means, e.g., live audio session, live audio and video session, live 3D session, etc. For a live 3D session, the user may be able to use a virtual augmentation glasses used for playing video games, etc., and engage in the live interactive therapy and counseling session with the chabot in 3D manner as if they are in a therapy room together. To engage in the therapy and counseling session, the user may open up the mental health monitoring app on the screen of the mental health monitoring device 10. Upon the user's opening of the mental health monitoring app, the chatbot appears and interacts with the user by asking simple questions, e.g., Hello John, How are you feeling today?, etc., as shown in the one-on-one therapy and counseling session via text 300A as shown in FIG. 3A. Alternatively, the chatbot may alert the user or initiate the therapy and counseling session with the user if the mental health monitoring device 10 has determined that the severity of the user's current or potential mental health condition poses a high risk—the severity may be categorized as a low risk, medium risk and high risk. The risk may be determined with respect to the user and/or the persons near the user. The chatbot is capable of conducting the therapy and counseling session as if the user is interacting with a mental health expert since the chatbot utilizes an AI based dialogue conversational engine, AI based mental health and wellbeing question engine, or AI based mental health and wellbeing response engine.

FIGS. 4A-B illustrate example screen shots of a main dashboard 400 the mental health monitoring device 10 in accordance with the present disclosure. FIG. 4A shows a main dashboard 400 of the mental health monitoring app 10A, listing the user data monitored and tracked by the mental health monitoring device 10. It will be understood by the person of ordinary skill in the art that content, display, types of activities shown in the main dashboard 400 are not limiting and for illustrative purposes only, and thus, may vary. For example, it may include a medication in-take data so as to inform the user of any missed or over-used medication. FIG. 4B shows a sleep data subpage 405A, displaying detailed sleep data 405B, e.g., a number of hours of sleep, hours in bed, heart rates, breathing rates, a number of bathroom visits, and a graph 405C showing the quality of sleep, etc. It will be understood by the person of ordinary skill in the art that a type, depth, content, and display of the detailed sleep data shown in the sleep data subpage are not limiting and for illustrative purposes only, and thus may include more or less, different sleep data. For example, the subpage may include the last activity performed by the user or the last person the user communicated with before falling asleep.

FIG. 5 illustrates a screen shot of a comprehensive wellbeing subpage 500 of the mental health monitoring device 10 in accordance with the present disclosure. The comprehensive wellbeing subpage 500 may include a variety of self-help, for example, supportive therapy session 505 (e.g., a self-help therapy session), therapeutic music 510 (e.g., mindfulness, meditation, or self-help video or audio), journal 515, a group chat connection 520, and a telehealth feature 525. The comprehensive wellbeing subpage 500 is discussed fully above with reference to FIG. 2, and thus, further description is omitted herein. However, it will be understood by the person of ordinary in the art that the self-help displayed on the comprehensive wellbeing subpage 500 is not limiting and for illustrative purpose only, and thus, may include different types of self-help appropriate for the identified symptom(s) and potential mental health condition. For example, the comprehensive wellbeing subpage 500 may include a list of medical or research books that may help the user to understand and learn about his/her current or potential mental health condition.

FIG. 6 illustrates a screen shot of a self-help recommendation plan 600 based on the collected user data displayed on the screen of the mental health monitoring device 10 in accordance with the present disclosure. The self-help recommendation plan 600 may be targeted to cope, manage, and/or deal with the identified symptom(s) and potential mental health condition, and thus, may vary depending on the identified symptom(s) and mental health condition. As such, it would be understood by the person of ordinary skill in the art that the self-help recommendation plan 600 may include different recommendations for different mental health conditions.

FIG. 7 illustrates a screen shot showing an online group chat 700 accessed via the mental health monitoring device 10 in accordance with the present disclosure. The user may select from a list of group chat channels available on a group chat subpage. In FIG. 7, the group chat channel 700 selected by the user is for stress management. Each of the group chat channels is focused on a specific mental health condition or disorder and the user may select a channel that is most appropriate and/or comfortable for his/her symptom(s) and potential mental health condition determined by the mental health monitoring device 10. Further, each channel may be vetted by the mental health monitoring device 10 so as to ensure the user safety and obtain a desired effect from participating in the selected group chat channel.

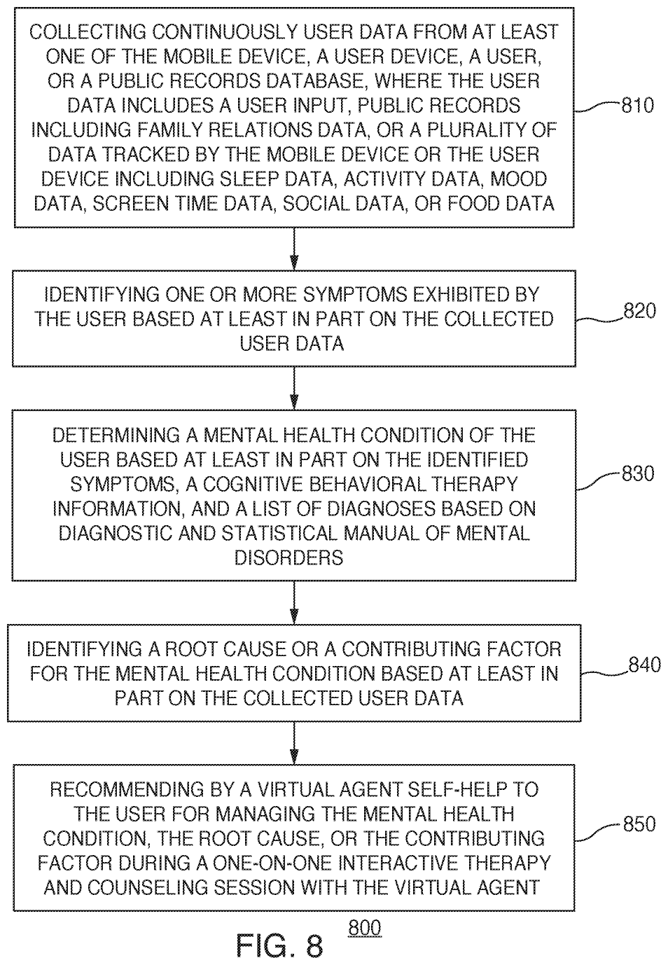

FIG. 8 is a flow chart for a method 800 for monitoring mental health using a mobile device in accordance with the present disclosure. The method 800 may be performed by any mobile device which may be a user equipment (e.g., a smart phone) or any component therein capable of performing the functions and operations of the mental health monitoring device as described with reference to FIGS. 1-7. As such, the mobile device may be understood to refer to the mental health monitoring device in accordance with the present disclosure.

At 810, the mental health monitoring device collect continuously user data from at least one of the mobile device, a user device, a user, or a public records database. The user data may include a user input, public records including family relations data, or a plurality of data tracked by the mobile device or the user device comprising sleep data, activity data, mood data, screen time data, social data, or food data. The user devices may include a wearable device for monitoring or tracking at least one of the tracked data. The virtual agent may be a chatbot utilizing at least one of an AI based dialogue conversational engine, AI based mental health and wellbeing question engine, or AI based mental health and wellbeing response engine. The one-on-one interactive therapy and counseling session may include at least one of a text-only therapy and counseling session, a live audio therapy and counseling session, or a live video therapy and counseling session with the chatbot. A mood of the user is determined based on the mood data using one or more AI facial recognition model, AI voice based model, or AI text based sentiment mode. The social data is used to determine a potential mental health condition of the user based at least in part on a time spent by the user on a social media application, a phone usage, or a usage of the mental health monitoring device. In some examples, collecting continuously the user data may include implementing a mental health monitoring application in the mobile device, receiving an authorization from the user to access the user data from the mobile device and the user device, and providing a mental health questionnaire including a plurality of questions customized to the collected user data and tailored to obtain information related to the mental health condition of the user, the information including a type or severity of the mental health condition.

At 820, the mental health monitoring device may identify one or more symptoms exhibited by the user based at least in part on the collected user data.

At 830, the mental health monitoring device may determine a potential mental health condition of the user based at least in part on the identified symptom, a cognitive behavioral therapy information, and a list of diagnoses based on diagnostic and statistical manual of mental disorders. In some examples, determining the mental health condition of the user may include determining a level of severity of the mental health condition, where the level of severity comprises a low risk, a medium risk, or a high risk, and alerting the user of the mental health condition in case of determining the level of severity being at the high risk immediately upon the determination.

At 840, the mental health monitoring device may identify a root cause or a contributing factor for the mental health condition based at least in part on the collected user data.

At 850, the mental health monitoring device recommend, by a virtual agent, self-help to the user for managing the mental health condition, the root cause, or the contributing factor during a one-on-one interactive therapy and counseling session with the virtual agent. In some examples, recommending, by the virtual agent, self-help to the user may include directing the user to a mental health expert for a granular diagnosis and therapy in an online platform via the mental health monitoring application. The mental health questionnaire may use a patient health questionnaire—9 module. In some examples, recommending, by the virtual agent, self-help to the user may include displaying on a display screen of the mobile device a page for each of the tracked data and/or displaying on a display screen of the mobile device a page for recommended self-help including meditation, supportive therapy for the contributing factor or the root cause related to the determined mental health condition, relaxation techniques, or an audio session for inducing positive thoughts, peace, or stress relief.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

What is claimed is:

1. A method for monitoring mental health using a mobile device, comprising:
collecting continuously user data from at least one of the mobile device, a user device, a user, or a public records database, wherein the user data comprises a user input, public records including family relations data, or a plurality of data tracked by the mobile device or the user device comprising sleep data, activity data, mood data, screen time data, social data, or food data;
identifying one or more symptoms exhibited by the user based at least in part on the collected user data;
determining a mental health condition of the user based at least in part on the identified symptoms, a cognitive behavioral therapy information, and a list of diagnoses based on diagnostic and statistical manual of mental disorders;
identifying a root cause for the mental health condition based at least in part on the collected user data; and
recommending, by a virtual agent, self-help to the user for managing the mental health condition and the root cause during a one-on-one interactive therapy and counseling session with the virtual agent.

2. The method of claim 1, wherein identifying the root cause comprises identifying a contributing factor for the mental health condition.

3. The method of claim 1, wherein collecting continuously user data comprises:
implementing a mental health monitoring application in the mobile device;
receiving an authorization from the user to access the user data from the mobile device and the user device; and
providing a mental health questionnaire including a plurality of questions customized to the collected user data and tailored to obtain information related to the mental health condition of the user, the information including a type or severity of the mental health condition.

4. The method of claim 3, wherein recommending, by the virtual agent, self-help to the user comprises directing the user to a mental health expert for a granular diagnosis and therapy in an online platform via the mental health monitoring application.

5. The method of claim 3, wherein the mental health questionnaire uses a patient health questionnaire—9 module.

6. The method of claim 1, wherein the user devices comprises a wearable device for monitoring or tracking at least one of the tracked data.

7. The method of claim 1, wherein the virtual agent is a chatbot utilizing at least one of an artificial intelligence (AI) based dialogue conversational engine, AI based mental health and wellbeing question engine, or AI based mental health and wellbeing response engine.

8. The method of claim 6, wherein the one-on-one interactive therapy and counseling session comprises at least one of a text-only therapy and counseling session, a live audio therapy and counseling session, or a live video therapy and counseling session with the chatbot.

9. The method of claim 1, wherein recommending, by the virtual agent, self-help to the user comprises displaying on a display screen of the mobile device a page for each of the tracked data.

10. The method of claim 1, wherein recommending, by the virtual agent, self-help to the user comprises displaying on a display screen of the mobile device a page for recommended self-help including at least one of supportive therapy sessions, therapeutic music, journal, or a connection to a group chat focused on the determined mental health condition.

11. The method of claim 1, wherein determining the mental health condition of the user comprises:
determining a level of severity of the mental health condition, wherein the level of severity comprises a low risk, a medium risk, or a high risk; and
alerting at least one of the user or a mental health expert of the mental health condition based at least part on a determination that the level of severity is at the high risk, immediately upon the determination.

12. The method of claim 1, wherein a mood of the user is determined based on the mood data using one or more artificial intelligence (AI) facial recognition model, AI voice based model, or AI text based sentiment model.

13. The method of claim 1, wherein the social data is used to determine the mental health condition of the user based at least in part on a time spent by the user on a social media application, a phone, or the mental health monitoring device.

14. The method of claim 1, wherein an artificial intelligence based prediction of a future mental health condition of the user is made based on food habits of the user.

15. An apparatus for monitoring mental health, comprising:
a processor;
memory in electronic communication with the processor; and
instructions stored in the memory and executable by the processor to cause the processor to:
collect continuously user data from at least one of the mobile device, a user device, a user, or a public records database, wherein the user data comprises a user input, public records including family relations data, or a plurality of data tracked by the mobile device or the user device comprising sleep data, activity data, mood data, screen time data, social data, or food data;
identify one or more symptoms exhibited by the user based at least in part on the collected user data;
determine a mental health condition of the user based at least in part on the identified symptoms, a cognitive behavioral therapy information, and a list of diagnoses based on diagnostic and statistical manual of mental disorders;
identify a root cause of the mental health condition based at least in part on the collected user data; and
recommend, by a virtual agent, self-help to the user for managing the mental health condition and the root cause during a one-on-one interactive therapy and counseling session with the virtual agent.

16. The apparatus of claim 15, wherein the instructions cause the processor to collect continuously user data comprises instructions for the processor to:
implement a mental health monitoring application in the mobile device;
receive an authorization from the user to access the user data from the mobile device and the user device; and
provide a mental health questionnaire including a plurality of questions customized to the collected user data and tailored to obtain information related to the mental health condition of the user, the information including a type or severity of the mental health condition.

17. The apparatus of claim 15, wherein the virtual agent is a chatbot utilizing at least one of an artificial intelligence (AI) based dialogue conversational engine, AI based mental health and wellbeing question engine, or AI based mental health and wellbeing response engine.

18. The apparatus of claim 17, wherein the one-on-one therapy and counseling session comprises at least one of a text-only therapy and counseling session, a live audio therapy and counseling session, or a live video therapy and counseling session with the chatbot.

19. The apparatus of claim 15, wherein the instructions to cause the processor to determine the mental health condition of the user comprises instructions to cause the processor to:
determine a level of severity of the mental health condition, wherein the level of severity comprises a low risk, a medium risk, or a high risk; and
alert at least one of the user or a mental health expert of the mental health condition in case of determining the level of severity being at the high risk, immediately upon the determination.

20. A non-transitory computer-readable medium storing code for monitoring mental health, the code comprising instructions executable by a processor to:
collect continuously user data from at least one of the mobile device, a user device, a user, or a public records database, wherein the user data comprises a user input, public records including family relations data, or a plurality of data tracked by the mobile device or the user device comprising sleep data, activity data, mood data, screen time data, social data, or food data;
identify one or more symptoms exhibited by the user based at least in part on the collected user data;
determine a mental health condition of the user based at least in part on the identified symptoms, a cognitive behavioral therapy information, and a list of diagnoses based on diagnostic and statistical manual of mental disorders;
identify a root cause of the mental health condition based at least in part on the collected user data; and
recommend, by a virtual agent, self-help to the user for managing the mental health condition and the root cause during a one-on-one interactive therapy and counseling session with the virtual agent.
